(19) United States
(12) Patent Application Publication
(10) Pub. No.: US 2015/0126918 A1
(43) Pub. Date: May 7, 2015

(54) UNIVERSAL SPINE BRACE WITH CABLE TENSIONING SYSTEM

(71) Applicant: DeRoyal Industries, Inc., Powell, TN (US)

(72) Inventors: Michael D. Modglin, Braselton, GA (US); Karen M. Clements, Knoxville, TN (US); Charles J. French, III, Lenoir City, TN (US); Sarah O. Davis, Gonzales, LA (US); Gregory S. Hodge, Knoxville, TN (US)

(21) Appl. No.: 14/524,612

(22) Filed: Oct. 27, 2014

Related U.S. Application Data
(60) Provisional application No. 61/901,169, filed on Nov. 7, 2013.

(51) Int. Cl. A61F 5/02

(52) U.S. Cl. CPC A61F 5/028; A61F 2250/001
USPC 602/19

(57) ABSTRACT

An adjustable and tensionable spinal brace, including a pair of conformable waist belt and a cable tensioning system releasably and adjustably attachable to the waist belt. The cable tensioning system includes at least one cable, a pair of cable spreaders, and one or more cable take-ups. The waist belt is initially snugly installable around the waist of the user by overwrapping the anterior ends, with portions of the cable tensioning system adjacent the spine of the user. The cable tensioning system is operable to enable micro adjustment of compressive forces applied via the brace to impart a desired compression or tension about the waist of the user.

### CROSS-REFERENCE TO RELATED APPLICATION

This application claims priority to U.S. Provisional Application No. 61/901,169, filed Nov. 7, 2013, and entitled Universal Spine Brace With Cable Tensioning System, incorporated by reference herein in its entirety.

### FIELD

This disclosure relates to the field of orthopedic bracing. More particularly, this disclosure relates to adjustable lumbar sacral spinal braces having a cable tensioning system.

### BACKGROUND

Improvement is desired in the provision of soft and flexible braces for supporting the thoraco-lumbro-sacral spine. While conventional lumbar belt type braces provide support, improvement is desired in providing a soft and flexible lumbo-sacral brace that is adjustable to fit a wide range of waist sizes circumferentially, and which enables micro adjustment of the compressive force applied via the brace.

The above and other needs are met by an adjustable and tensionable spinal brace, including a pair of conformable waist belts and a cable tensioning system releasably and adjustably attachable to the waist belts. Braces according to the disclosure enable improved fit and tensioning, and enable micro adjustment of the compressive force applied via the brace.

### SUMMARY

The present disclosure relates to an adjustable and tensionable spinal brace.

In one aspect, the brace includes a waist belt having a pair of belts, each having an anterior end and a posterior end. The waist belt is installable about a waist of a user so that anterior end of each belt are connected together and the posterior end of the each belt is adjacent the spine of the user. A cable tensioning system is releasably and adjustably attachable to the waist belt. The cable tensioning system has a pair of cables, a pair of cable spreaders, and a pair of cable take-ups.

In another aspect, the brace includes a waist belt installable about a waist of a user so that anterior ends of the belt are connected together, and a cable tensioning system releasably and adjustably attachable to the waist belt. The cable tensioning system includes at least one cable, a pair of cable spreaders, and a cable take-up.

The brace is initially snugly installable around the waist of the user by overwrapping the anterior ends, with portions of the cable tensioning system adjacent the spine of the user. The cable tensioning system is operable to enable micro adjustment of compressive forces applied via the brace to impart a desired compression or tension about the waist of the user.

### BRIEF DESCRIPTION OF THE DRAWINGS

Further advantages of the disclosure are apparent by reference to the detailed description when considered in conjunction with the figures, which are not to scale so as to more clearly show the details, wherein like reference numbers indicate like elements throughout the several views, and wherein:

FIG. 1 is a front perspective view of an adjustable brace according to the disclosure.

FIG. 2 is a rear perspective view of the brace of FIG. 1.

FIG. 3 is a front plan view of the brace of FIG. 1.

FIG. 4 is a rear plan view of the brace of FIG. 1.

FIG. 5 is a partially exploded view of the brace of FIG. 1.

FIG. 6 is a front view of a cable tensioning system of the brace of FIG. 1.

FIG. 7 is a rear view of the cable tensioning system of FIG. 6.

FIG. 8 is a close-up view of a cable spreader component of the cable tensioning system of FIG. 7.

FIG. 9 is a close-up view of a cable take-up component of the cable tensioning system of FIG. 7.

FIG. 10 is a front plan view of an alternate embodiment of an adjustable brace according to the disclosure.

FIG. 11 is a close-up view of a portion of the adjustable brace of FIG. 10.

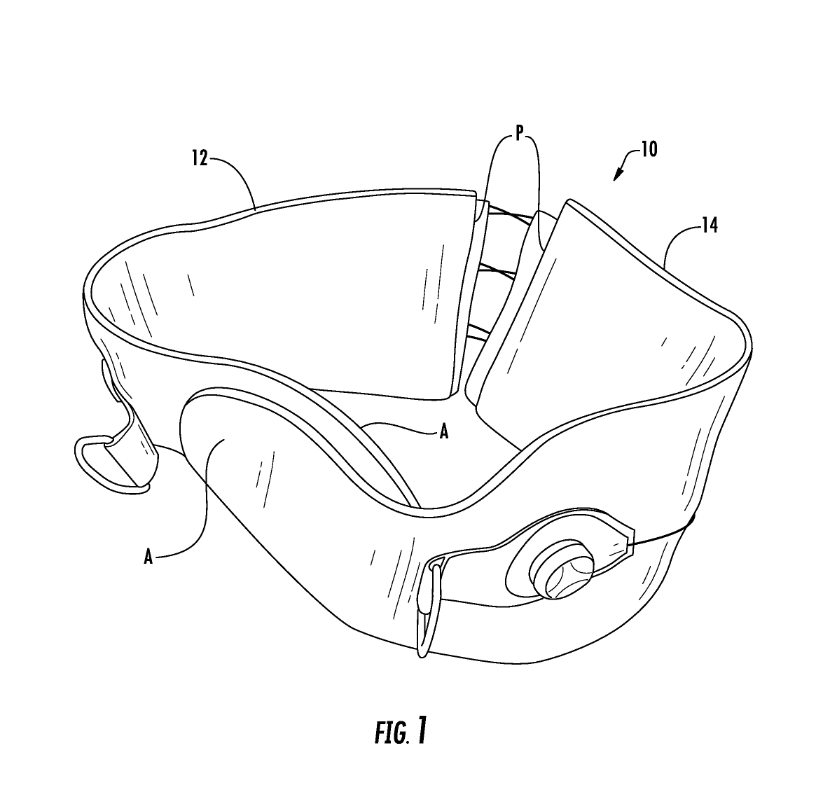

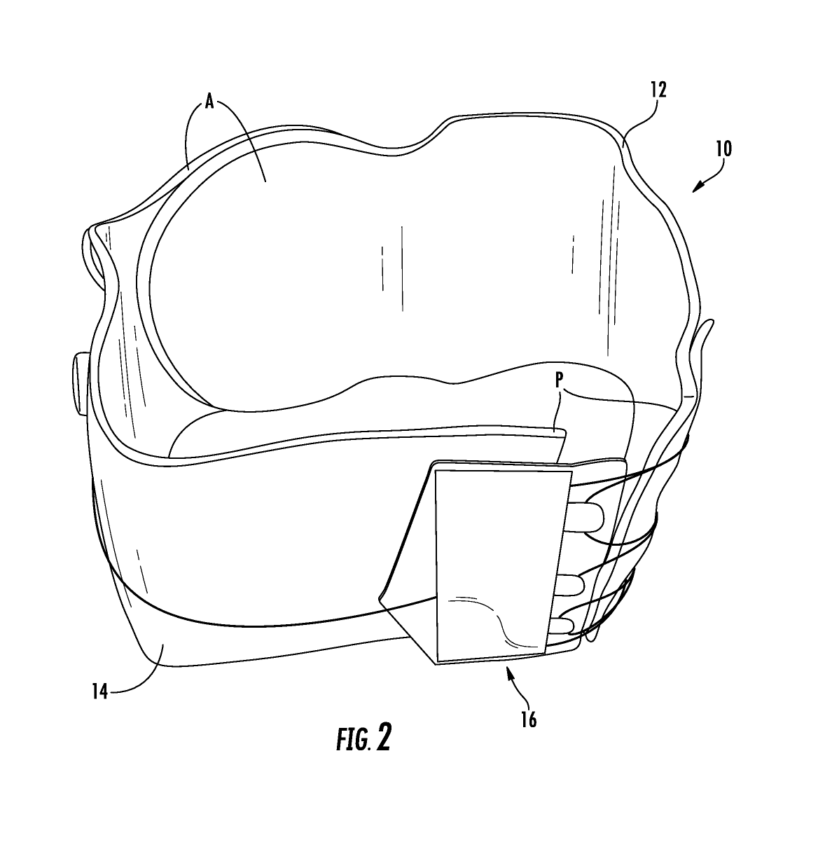

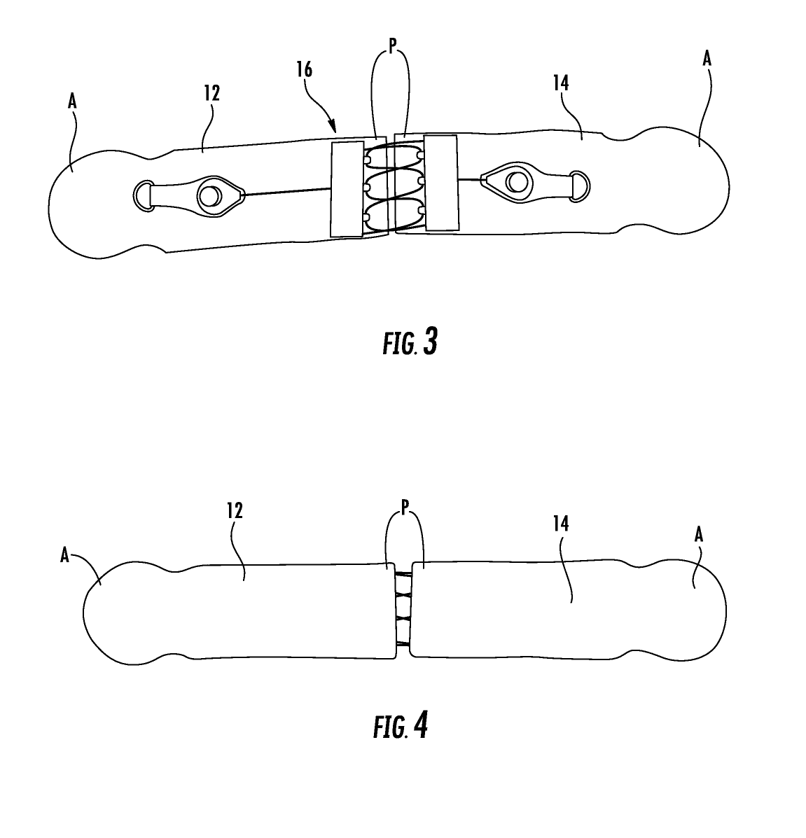

### DETAILED DESCRIPTION

With reference to the drawings, there is shown an adjustable length lumbar sacral brace 10 that enables micro adjustment of the tension of the brace. The brace 10 includes a pair of conformable waist belts 12 and 14 and a cable tensioning system 16 releasably and adjustably attachable to the waist belts 12 and 14. Posterior ends P of the belts 12 and 14 are positioned end to end, slightly spaced apart, and anterior ends A of the belts 12 and 14 are overlapped and secured to one another about the waist of the user.

In use, the brace 10 is initially snugly installed around the waist of the user by overwrapping the anterior ends A of the waist belts 12 and 14, with portions of the cable tensioning system spanning between the posterior ends P of the belts, The posterior ends P of the belts are adjacent the spine of the user and may be spaced from one another, touching end to end, or overlapped. The cable tensioning system 16 enables micro adjustment of the compressive force applied via the brace 10 to impart a desired compression or tension about the waist of the user.

The belts 12 and 14 are made of an elongate, flexible, and conformable material such as a soft fabric surrounding a batting or padding, such as a soft foam or spacer fabric. The belts 12 and 14 preferably include a receiver material on their front surface, such as a loop material, and located for adjustable and releasable engagement of the cable tensioning system 16 to the belts 12 and 14.

The belts 12 and 14 may include padding or a rigid stay or the like to protect from the cable tensioning system 16 cutting into or applying excessive pressure through the belts 12 and 14. The anterior ends A of the belts 12 and 14 preferably include mating hook and loop material or other cooperating fastening structure to enable adjustable overlapping of the anterior ends A to enable initial fitting of the brace 10 to the user and to maintain the anterior ends A together.

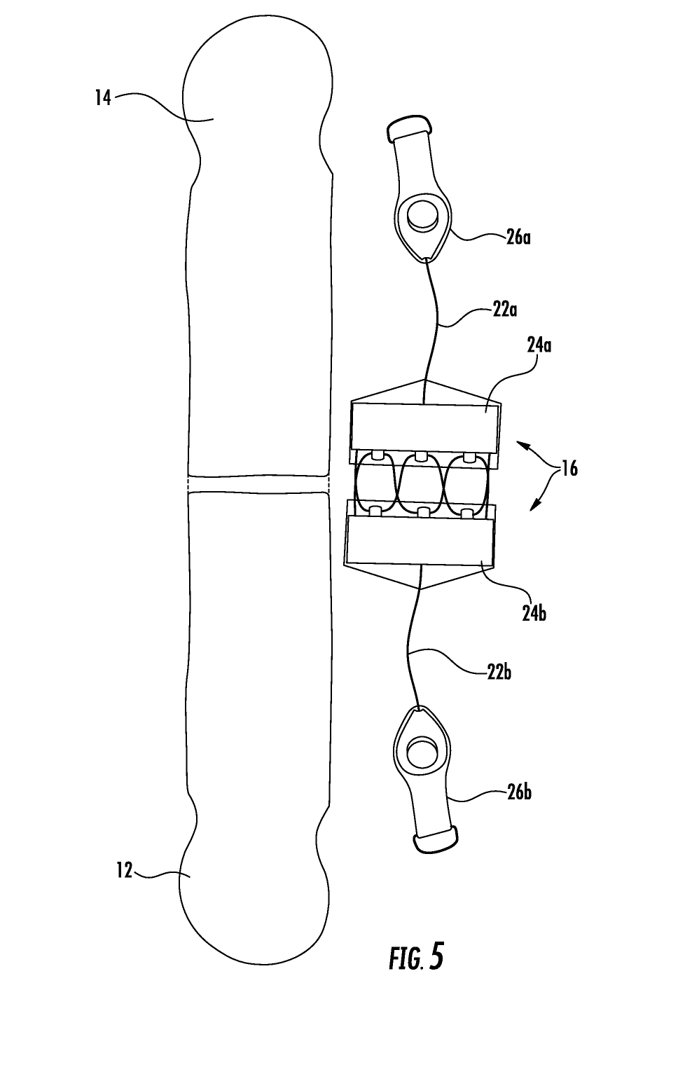

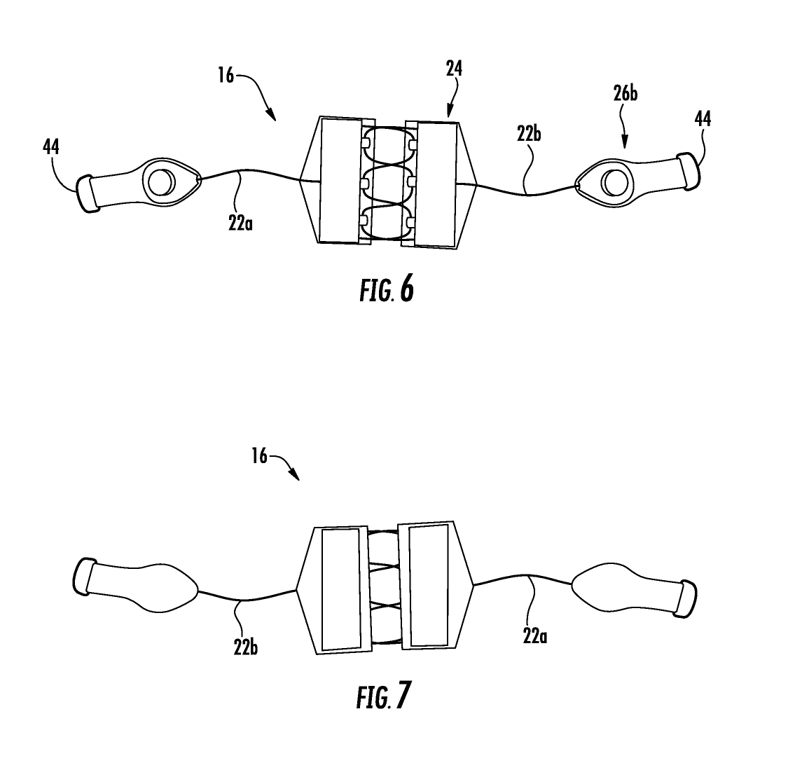

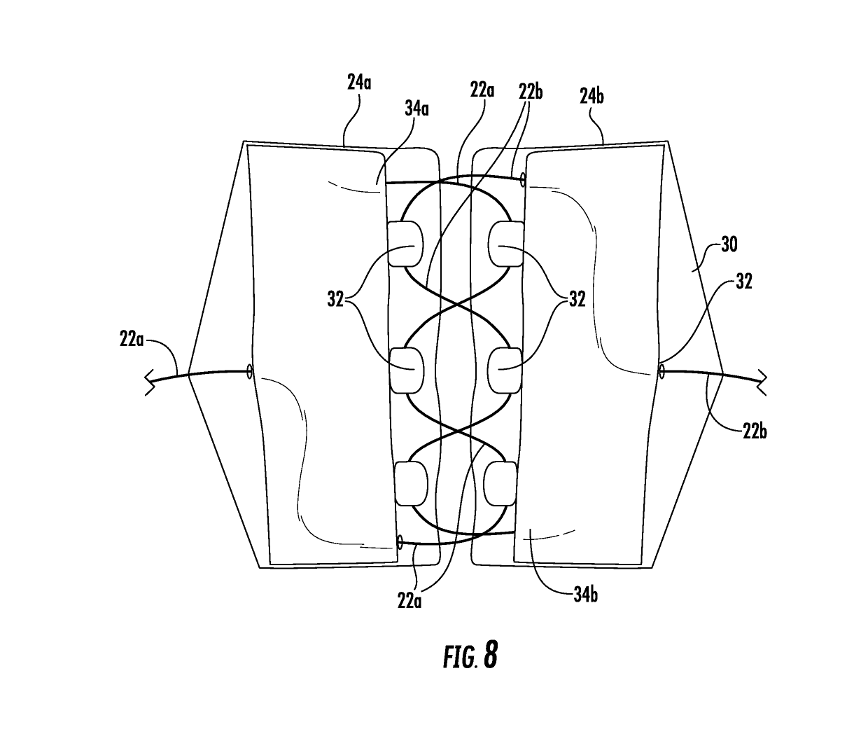

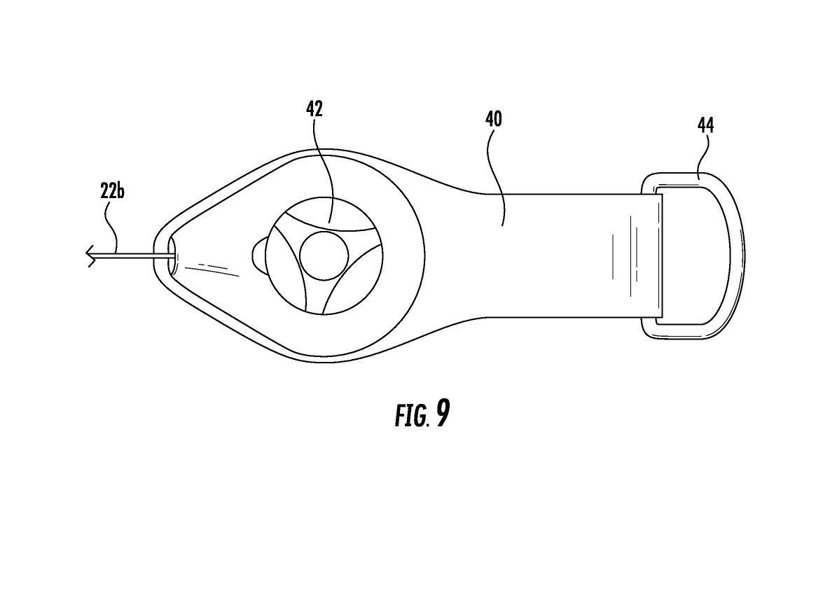

Also, a single belt may be used instead of the two belts, with the tensioning system 16 located in the same manner as if two belts were utilized. However, this configuration is more desirable if the belt is substantially custom sized to the user so as to avoid undesired bunching in the back. The two belt configuration is preferred for non-custom situations as it enables a single size to readily be adapted to a wide range of waist sizes. The dashed lines added to FIG. 5 represent the option of having a single belt instead of two belts.

The cable tensioning system 16 is adjustably and releasably attachable - to the waist belts 12 and 14. For example, as described below, components of the cable tensioning system 16 include a hook material or the like thereon for cooperating with the receiver material on the front surfaces of the belts 12 and 14.

The cable tensioning system 16 is configured to enable micro adjustment of the compressive force applied via the brace 10 to impart a desired compression or tension about the waist of the user. In a preferred embodiment, the cable tensioning system 16 includes a pair of cables 22a and 22b, a pair of cable spreaders 24a and 24b, and a pair of cable take-ups 26a and 26b.

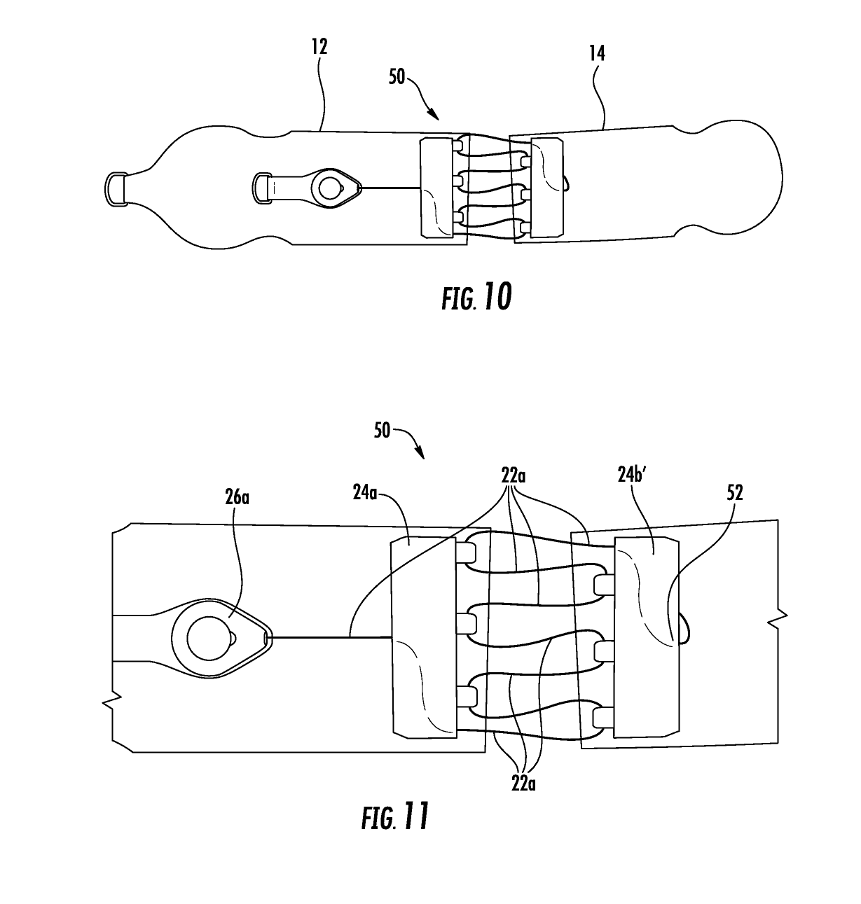

While the system 16 is shown having a pair of the cable take-ups 26a and 26b, alternatively, a single cable may be used in place of two cables, with each end of the single cable being connected to one of the cable take-ups 26a and 26b. Also, a single one of the take-ups 26a or 26b may be utilized, with the other one of the cable take-ups 26a or 26b replaced with a cable fixation point. An embodiment having a single take-up is shown in FIGS. 10 and 11.

The cable spreaders 24a and 24b may be identical and each provided as by a base 30 having a plurality of cable guides 32 thereon through which the cables 22a and 22b are trained so as to increase the surface area over which the cables 22a and 22b are trained. This configuration is desirable to avoid bunching and to more uniformly distribute the force at the spine. The backside of the bases 30 includes a hook material or the like for adjustably and releasably securing the cable spreaders 24a and 24b to the belts 12 and 14. In the embodiment shown, the cables 22a and 22b are trained back and forth between the guides 32 of one of the cable spreaders 24a to the other cable spreader 24b. A terminal end of the cable 22a is secured by a stop 34a, and a terminal end of the cable 24 is secured by a stop 34b. However, it will be understood that the cables 22a and 22b, or just a single cable or multiple cables, may be routed in other configurations.

The cable take-ups 26a and 26b may each be provided as by a base 40, such as a fabric base, onto which is mounted a take-up device such as a cable reel 42. Suitable cable reel devices are available under the name BOA from Boa Technology, Inc. of Denver, Colo., and described in U.S. Pat. Nos. 7,954,204 and 7,992,261, incorporated by reference in their entireties. The backside of the bases 40 includes the hook material or the like for adjustably and releasably securing the cable take-ups 26a and 26b to the belts 12 and 14. A D-ring 44 or other handle device may be included on the base 40 for grasping to pre-tension the cables 22a and 22b when the bases 40 are located on the belts 12 and 14. Thereafter, the cable reels (or spook or other devices that enable take-up or adjustment of the cable length such as hook material or the like connected to the cables and attachable to the belts) may be used to desirably tension the cables 22a and 22b.

The brace 10 is installed on the waist of the user by initially securing the belts 12 and 14 around the waist of the user and overlapping and securing the anterior ends A so that the belts 12 and 14 are snugly about the waist of the user. The tensioning system 16 is preferably initially engaged onto the belts 12 and 14, with the cable spreaders 24 at the posterior ends P of the belts 12 and 14. Next, with the cables 22a and 22b are adjusted to permit the desired positioning of the cable take-ups 26a and 26b, the take-ups 26a and 26b are positioned, as by pulling on the D-rings 44 to position the take-ups 26a and 26b near the anterior ends A of the belts 12 and 14, and securing the base 40 of the take-ups 26a and 26b to the receiver material of the belts 12 and 14, preferably with the cables 22a and 22b taut.

However, it will be understood that the entire surface of the belts 12 and 14 can be configured for adjustable and releasable engagement of the cable tensioning system 16 to the belts 12 and 14. The take-ups 26a and 26b are then manipulated to apply any additional desired tension to the cables 22a and 22b. For example, in the event one or both of the take-ups are provided by cable reels, the reels may be operated to windup the cables 22a and 22b to further tension the cables 22a and 22b. In this manner, the brace 10 provides three levels of tensioning. A first tension is applied to the belts 12 and 14 by installing them. Further tension may be applied when positioning the cable take-ups 26a and 26b. Once the take-ups 26a and 26b are desirably located, winding or unwinding of the cable 22a, the cable 22b, or both using one or both of the take-ups 26a and 26b enables desired adjustment of the tension of the cable 22a and 22b.

As mentioned above, a single one of the take-ups 26a or 26b may be utilized, with the other one of the cable take-ups 26a or 26b replaced with a cable fixation point. As shown in FIGS. 10 and 11, a brace 50 is shown having just the take-up 26a with a modified cable spreader 24b′ configured to terminate the single cable 22a at a stop 52.

While the disclosure describes the construction of a lumbo-sacral belt or brace, it will be appreciated that rigid or semi-rigid components may be added anteriorly, posteriorly, or laterally to the described brace to increase stability into the thoracic or even the cervical spine so that a patient can be treated with one brace as they progress through different stages of treatment or healing.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and As practical application, and to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

1. An adjustable and tensionable spinal brace, comprising:
a waist belt comprising a pair of belts, each having an anterior end and a posterior end, wherein the waist belt is installable about a waist of a user so that anterior end of each belt are connected together and the posterior end of the each belt is adjacent the spine of the user; and
a cable tensioning system releasably and adjustably attachable to the waist belt, the cable tensioning system comprising a pair of cables, a pair of cable spreaders, and a pair of cable take-ups,
wherein the waist belt is initially snugly installable around the waist of the user by overwrapping the anterior ends, with portions of the cable tensioning system adjacent the spine of the user, and the cable tensioning system is operable to enable micro adjustment of compressive forces applied via the brace to impart a desired compression or tension about the waist of the user.

2. An adjustable and tensionable spinal brace, comprising:
a waist belt installable about a waist of a user so that anterior ends of the belt are connected together; and
a cable tensioning system releasably and adjustably attachable to the waist belt, the cable tensioning system comprising at least one cable, a pair of cable spreaders, and a cable take-up,
wherein the waist belt is initially snugly installable around the waist of the user by overwrapping the anterior ends, with portions of the cable tensioning system adjacent the spine of the user, and the cable tensioning system is operable to enable micro adjustment of compressive forces applied via the brace to impart a desired compression or tension about the waist of the user.

3. The brace of claim 2, wherein the cable take-up is a cable reel.

4. The brace of claim 2, wherein the cable spreaders are each located adjacent the spine of the user.

5. The brace of claim 2, wherein the waist belt: comprises a pair of belts, each having an anterior end and a posterior end, wherein the waist belt is installable about a waist of a user so that anterior end of each belt is connected together and the posterior end of the each belt is adjacent the spine of the user.
